(19) United States
(12) Patent Application Publication
(10) Pub. No.: US 2024/0165099 A1
(43) Pub. Date: May 23, 2024

(54) METHODS FOR THE TREATMENT AND PREVENTION OF DISEASES OR INFECTIONS WITH MCP-1 INVOLVEMENT BY ADMINISTRATION OF TAFENOQUINE

(71) Applicant: 60 DEGREES PHARMACEUTICALS LLC, Washington, DC (US)

(72) Inventor: Geoffrey S. DOW, Washington, DC (US)

(73) Assignee: 60 DEGREES PHARMACEUTICALS LLC, Washington, DC (US)

(21) Appl. No.: 18/375,070

(22) Filed: Sep. 29, 2023

Related U.S. Application Data
(60) Provisional application No. 63/411,654, filed on Sep. 30, 2022.

(51) Int. Cl. A61K 31/4706; A61K 45/06; A61P 31/14

(52) U.S. Cl. CPC A61K 31/4706; A61K 45/06; A61P 31/14

(57) ABSTRACT

Methods for treating or preventing a disease or infection with MCP-1 involvement, or related symptoms, using an effective amount of tafenoquine are disclosed. Compositions for treating or preventing a disease or infection with MCP-1 involvement, or related symptoms, using an effective amount of tafenoquine are disclosed. Kits including tafenoquine are disclosed.

### CROSS-REFERENCE TO RELATED APPLICATIONS

This application claims the benefit of U.S. Provisional Application Ser. No. 63/411,654, filed Sep. 30, 2022, which is hereby incorporated by reference herein in its entirety, including any figures, tables, or drawings.

### BACKGROUND OF THE INVENTION

Monocyte chemotactic protein 1 (MCP-1) is one of the major chemokines involved in migration of monocytes and macrophages to sites of infection. As such, it is a key inflammatory protein and a point of regulation of the inflammatory response. There is an urgent need for methods of treating or preventing diseases or infections with MCP-1 involvement. In particular, there is an urgent need for methods of treating or preventing diseases or infections in which the cytokine MCP-1 is known to be upregulated.

### SUMMARY OF THE INVENTION

The present invention concerns the use of long half-life 8-aminoquinolines for the treatment or prevention of diseases or infections with MCP-1 involvement. In particular embodiments, MCP-1 expression is increased in subjects with said disease or infection compared to subjects without said disease or infection. In some embodiments, the long half-life 8-aminoquinoline is tafenoquine.

One aspect of the invention pertains to compositions and methods for administering a long half-life 8-aminoquinoline that meet the unmet medical need for preventing or treating diseases and infections with MCP-1 involvement. In particular embodiments, MCP-1 expression is increased in subjects with said disease or infection compared to subjects without said disease or infection. In further embodiments, the method further comprises administering a second agent, such as a drug, to the human subject.

In particular embodiments, the long half-life 8-aminoquinoline is tafenoquine, or a pharmaceutically acceptable salt thereof. The long half-life 8-aminoquinoline may be administered to the human subject as at least one initial (loading) dose. The method may include detecting the presence of a disease or infection with MCP-1 involvement prior to administration of the long half-life 8-aminoquinoline. In one specific embodiment, the disease or infection is generally known to be associated with elevated MCP-1. In other embodiments, the level of MCP-1 expression is determined prior to administration of the long half-life 8-aminoquinoline. Any suitable method for diagnosing or testing can be used, and many such methods are well known in the art, including involving nucleic acid or protein assays.

For both methods of treatment of diseases and infections with MCP-1 involvement and methods of prevention of diseases and infections with MCP-1 involvement, the method comprises administering to said subject an effective amount of tafenoquine or a compound of Formula (I), a pharmaceutically-acceptable salt thereof, or a pharmaceutical composition comprising tafenoquine or a compound of Formula (I),

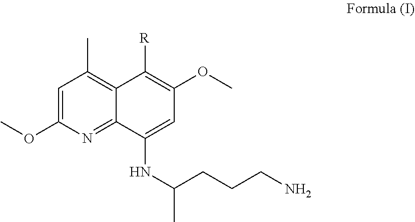

wherein R is any halogen-containing substituent of molecular weight 205. In another embodiment, the subject is a G6PD-normal human. In preferred embodiments, Formula (I) is tafenoquine. In other embodiments, the method comprises administering to said subject an effective amount of a second agent. In a further embodiment, the administration of tafenoquine or said compound of Formula (I) and administration of said second agent is concurrent. In yet other embodiments, the administration of said compound of Formula (I) and administration of said second agent is not concurrent. In even further embodiments, no second agent is administered.

In other embodiments, the second agent is selected from the group consisting of amikacin, an aminoglycoside, amoxicillin, amphotericin formulations, any drug approved by the Food and Drug Administration for treating or preventing bacterial viral, and/or fungal infections, any azole-containing anti-fungal drug, atovaquone, azithromycin, Bactrim, bedaquiline, a benzothiazinone, BTZ043, capreomycin, cefftriaxime, cefotaxime, cefuroxamine, clindamycin, clofazimine, corticosteroids, a cyclic peptide, cycloserine, delamanid, a diarylquinoline, echinocandin, ethambutol, ethionamide, fluconazole, flucytosine, a fluoroquinolone, an imidazopyridine amide, isoniazid, itraconazole, kanamycin, levofloxacin, linezolid, a macrolide, moxifloxacin, a nitroimidazole, an oxazolidinone, PA-824, para-aminosolicyclic acid, PBTZ169, posaconazole, prothionamide, pyrazinamide, Q203, quinine, rifampin, rifapentine, SQ-109, streptomycin, sulfa drugs, sutezolid, a thioamide, trimethoprimsulfamethoxazole, vancomycin, voriconazole, any anti-viral drug, remdesivir, favipiravir, chloroquine, hydroxychloroquine, a monoclonal antibody treatment, a steroid, COVID-19 convalescent plasma, casirivimab, imdevimab, bamlanivimab, baricitinib, interleukin-6 inhibitors, kinase inhibitors, tyrosine kinsase inhibitors, Tocilizumab, ivermectin, and any combination thereof.

In further embodiments said human has a coronavirus infection (e.g., SARS-CoV-2 infection) and/or has one or more risk factors for disease progresses selected from the group consisting of: age of 60 years old or older, obesity, diabetes, and heart disease. In particular embodiments, said human subject has COVID-19 disease and/or has one or more risk factors for disease progresses selected from the group consisting of: age of 60 years old or older, obesity, diabetes, and heart disease.

In other embodiments, said subject is symptomatic of a disease or infection with MCP-1 involvement prior to the first administration. In particular embodiments, the human subject is symptomatic for a coronavirus infection (e.g., SARS-CoV-2 infection) at the time of first administration.

In other embodiments, said subject is asymptomatic of a disease or infection with MCP-1 involvement prior to the first administration. In particular embodiments, the human subject is asymptomatic fora coronavirus infection (e.g., SARS-CoV-2 infection) at the time of first administration.

In further embodiments, the human subject has tested positive for a disease or infection with MCP-1 involvement at the time of the administration but is asymptomatic. In some embodiments, the human subject is asymptomatic for the coronavirus infection and/or has been diagnosed as coronavirus negative (e.g., SARS-CoV-2 negative) at the time of the administration. In other embodiments, the human subject has been exposed to coronavirus (e.g., SARS-CoV-2) or has had close contact with someone infected with the coronavirus (e.g., SARS-CoV-2).

In other embodiments, said disease or infection with MCP-1 involvement is latent prior to the first administration.

In other embodiments, said subject is at risk of contracting a disease or infection with MCP-1 involvement. In some embodiments, the method is for decreasing severity of a disease or infection with MCP-1 involvement and/or accelerating the recovery from a disease or infection with MCP-1 involvement, wherein the first administration is prior to potential exposure of bacterial, fungal, or viral infections (this may be referred to a method of pre-treatment). In particular embodiments, the method is for decreasing severity of COVID-19 disease and/or accelerating recovery of a subject from symptomatic COVID-19 disease, wherein the first administration is prior to said subject exposure or potential exposure to SARS-CoV-2.

In other embodiments, the method is for preventing or treating a disease or infection with MCP-1 involvement and associated morbidity and mortality in G6PD normal human subject occurring during or while in recovery from a corona viral infection (e.g., SARS-CoV-2 infection). In further embodiments, said human subject with corona viral infection has COVID-19 disease. In other embodiments, the method is for preventing or treating a disease or infection with MCP-1 involvement in G6PD normal subject with suspected COVID-19 disease. In further embodiments said subject with a corona viral infection (e.g., SARS-CoV-2 infection) has or is at risk of neutropenia. In other embodiments, said subject with COVID-19 disease has or is at risk of neutropenia.

In further embodiments, said human subject has or is at risk of neutropenia. In other embodiments, said subject who has or is at risk of neutropenia has hematologic malignancies. In even further embodiments said subject who has or is at risk of neutropenia has received chemotherapy, is a transplant recipient under immunosuppressive treatment, is HIV positive with low T-cell counts, is experiencing other infectious diseases in which the immune system is suppressed, is taking courses of immunosuppressive medication including corticosteroids, and/or is taking antibody treatments for chronic diseases. In other embodiments, the transplant subject is receiving a bone marrow transplant, a heamatopietic stem cell transplant, or a solid organ transplant. In further embodiments, said subject has or is at risk of neutropenia, and/or is a transplant subject receiving a bone marrow transplant or a haematopoietic stem cell transplant or a solid organ transplant. In further embodiments, administration of tafenoquine to said subject comprises administration up to 90 days prior to transplantation or initiation of immunosuppressive therapy. For example, this may allow any minor hematologic changes associated with tafenoquine administration to normalize prior to transplantation or initiation of immunosuppressive therapy.

In some embodiments, administration of tafenoquine comprises a dosing regimen of 200 mg/day for three days followed by 200 mg once weekly for as long as permitted by regulators. In other embodiments, administration of tafenoquine comprises the dose of tafenoquine as much as 399 mg at the same regimens as described herein, or multiple doses, such as up to 8 doses, as specified in Table 1 or Table 2.

In further embodiments, said human subject has at least one of the following conditions selected from the group consisting of: is at risk of catching respiratory virus during the winter season (and therefore of contracting secondary infections), is elderly, is a surgical subject, has a catheter or iv line, has diabetes, has obesity, has COPD, has kidney disease, and has cardiac conditions. In further embodiments the subject is a child.

Yet another embodiment of the invention is a kit comprising: (a) a means for testing for G6PD deficiency; (b) tafenoquine or a compound of Formula (I), a pharmaceutically-acceptable salt thereof, or a pharmaceutical composition comprising tafenoquine or a compound of Formula (I); and

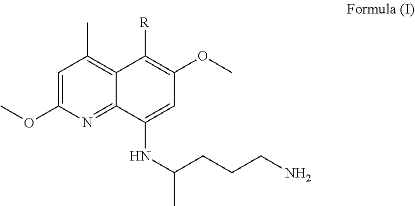

(c) instructions for use, wherein R is any halogen-containing substituent of molecular weight 205. In another embodiment, such a kit further comprises (d) an effective amount of a second agent. In particular embodiments said second agent is selected from the group consisting of amikacin, an aminoglycoside, amoxicillin, amphotericin formulations, any drug approved by the Food and Drug Administration for treating or preventing bacterial, viral, and/or fungal infections, any azole-containing anti-fungal drug, atovaquone, azithromycin, Bactrim, bedaquiline, a benzothiazinone, BTZ043, capreomycin, cefftriaxime, cefotaxime, cefuroxamine, clindamycin, clofazimine, corticosteroids, a cyclic peptide, cycloserine, delamanid, a diarylquinoline, echinocandin, ethambutol, ethionamide, fluconazole, flucytosine, a fluoroquinolone, an imidazopyridine amide, isoniazid, itraconazole, kanamycin, levofloxacin, linezolid, a macrolide, moxifloxacin, a nitroimidazole, an oxazolidinone, PA-824, para-aminosolicyclic acid, PBTZ169, posaconazole, prothionamide, pyrazinamide, Q203, quinine, rifampin, rifapentine, SQ-109, streptomycin, sulfa drugs, sutezolid, a thioamide, trimethoprimsulfamethoxazole, vancomycin, voriconazole, and any combination thereof.

In particular embodiments, said compound of Formula (I) is tafenoquine. In other particular embodiments, the long half-life 8-aminoquinoline is tafenoquine. In some embodiments, at least one dose of about 100 mg-600 mg of tafenoquine are administered. In a further embodiment, at least three doses of about 100 mg-600 mg of tafenoquine are administered. In another embodiment, at least seven doses of about 100 mg-600 mg of tafenoquine are administered. In other embodiments, administration is conducted according to the dosing regimen of any one of Tables 1-2 and/or according to any of the Examples. In other embodiments, no more than 10,800 mg is administered to said subject in a six-month period. In some embodiments, administration of tafenoquine comprises a dosing regimen of 200 mg/day for three days following by 200 mg once weekly for as long as permitted (e.g., permitted by regulators). In some embodiments, administration of tafenoquine comprises a dosing regimen of 200 mg/day for each of three days. In some embodiments, administration of tafenoquine comprises a dosing regimen of 200 mg/day for each of four days. In other related embodiments, a total of 600 mg of tafenoquine is administered over one to three days. In other related embodiments, a total of 800 mg of tafenoquine is administered over one to four days. In other embodiments, administration of tafenoquine comprises the dose of tafenoquine as much as 399 mg at the same regimens as described herein, or multiple doses, such as up to 8 doses, as specified in Table 1 or Table 2.

In other embodiments, the measured half-life of the compound of Formula (I) is at least three times greater than the measured half-life of primaquine. In other embodiments, the measured half-life or its metabolites in plasma or lung is at least three times longer than the measured half-life of primaquine in plasma or lung.

In other embodiments, said compound of Formula (I) is an 8-aminoquinoline with a measured half-life greater than primaquine. In other embodiments, the measured half-life of the 8-aminoquinoline or its metabolites in plasma or lung is longer than the measured half-life of primaquine in plasma or lung.

In other embodiments, administration is conducted such that gastro-intestinal disturbance in said subject is minimized. In other embodiments, administration is via sub-lingual and/or buccal route(s).

In further embodiments, Formula (I) is tafenoquine and administration comprises a loading dose every day for three days. In other embodiments, the long half-life 8-aminoquinoline is tafenoquine and administration comprises a loading dose every day for three days. In further embodiments the loading dose is 200 mg per day. In another embodiment, the human subject is administered 200 mg of tafenoquine once a day for three days and weekly 200 mg dose of tafenoquine. In another embodiment, the human subject is administered a total of 600 mg of tafenoquine over one to three days. In another embodiment, the human subject is administered a total of 800 mg of tafenoquine over one to four days. In certain embodiments, administration of oral dose of tafenoquine to the human subject is initiated once the human subject has recovered to the point where taking oral medications becomes feasible and wherein the dosing regimen is a loading dose for three days, followed by weekly doses for as long as permitted (e.g., by regulators). In further embodiments, said loading dose is 200 mg/day and the weekly doses is 200 mg per week. A further embodiment, administration of tafenoquine is a dose of as much as 399 mg and administered according to the regimens described herein, or multiple doses, such as up to 8 doses, as specified in Table 1 or Table 2. In particular embodiments, administration of tafenoquine prophylaxis is a loading dose of 200 mg every day for three days (or a total loading dose of 600 mg over one to three days). For example if the 600 mg loading dose was completed but symptom progression or mechanical ventilation subsequently rendered oral dosing temporarily unfeasible for one week, and less than 14 days has passed since the end of the loading dose, tafenoquine prophylaxis could be re-initiated starting with a loading dose of 400 mg [200 mg/day for two days] then 200 mg weekly thereafter for as long as permitted by regulators; for example if the 600 mg loading dose was completed but symptom progression or mechanical ventilation subsequently rendered oral dosing temporarily unfeasible for one week, and less than 21 days has passed since the end of the loading dose, tafenoquine prophylaxis could be re-initiated starting with a loading dose of 600 mg [200 mg/day for three days] then 200 mg weekly thereafter for as long as permitted by regulators; for example if only 400 mg loading dose was completed but symptom progression or mechanical ventilation subsequently rendered oral dosing temporarily unfeasible for one week, and less than 7 days has passed since the end of the loading dose, tafenoquine prophylaxis could be re-initiated starting with a loading dose of 400 mg [200 mg/day for two days] then 200 mg weekly thereafter; and for example if only 200 mg loading dose was completed but symptom progression or mechanical ventilation subsequently rendered oral dosing temporarily unfeasible for two weeks, and less than 14 days has passed since the end of the loading dose, tafenoquine prophylaxis could be re-initiated starting with a loading dose of 600 mg [200 mg/day for three days] then 200 mg weekly thereafter for as long as permitted by regulators. In a further embodiment, administration of tafenoquine is a dose of as much as 399 mg and administered according to the regimens described herein, or multiple doses, such as up to 8 doses, as specified in Table 1 or Table 2.

In particular embodiments, method of treatment of a disease or infection with MCP-1 involvement comprises administering to a human subject about 600 mg of tafenoquine over one to three days (e.g., 200 mg/day for three days; 400 mg on Day 1 and 200 mg on Day 3; 200 mg provided three times in a 24 hour period; or 600 mg on Day 1). In further embodiments, said human subject has tested positive a disease or infection with MCP-1 involvement. In particular embodiments, the expression level of MCP-1 has been determined in said human subject. In further embodiments, the expression level of MCP-1 is higher in said human subject compared to the standard expression level of MCP-1 (e.g., the expression level of MCP-1 from a healthy subject or from a controlled sample).

In particular embodiments, method of treatment of a disease or infection with MCP-1 involvement comprises administering to a human subject about 800 mg of tafenoquine over one to four days (e.g., 200 mg/day for four days; 400 mg on Day 1 and 200 mg on Days 3 and 4; 200 mg provided three times in a 24 hour-period and 200 mg provided once on Day 2, 3, or 4; 600 mg on Day 1 and 200 on Day 4; 266.66 mg/day for three days). In further embodiments, said human subject has tested positive for a disease or infection with MCP-1 involvement. In particular embodiments, the expression level of MCP-1 has been determined in said human subject. In further embodiments, the expression level of MCP-1 is higher in said human subject compared to the standard expression level of MCP-1 (e.g., the expression level of MCP-1 from a healthy subject or from a controlled sample).

In some embodiments, tafenoquine would be administered to asymptomatic individuals. In some embodiments, administration of tafenoquine comprises a dosing regimen of 200 mg/day for three days following by 200 mg once weekly for as long as permitted by regulators. In other embodiments, administration of tafenoquine comprises the dose of tafenoquine as much as 399 mg at the same regimens as described herein, or multiple doses, such as up to 8 doses, as specified in Table 1 or Table 2.

In some embodiments, the long half-life 8-aminoquinoline is used to treat a patient with a disease or infection with MCP-1 involvement. In particular embodiments a dose of 200 mg is administered on the first, second [+/−one day], third [+/−one day], and tenth [+/−one day] days. In particular embodiments a dose of 200 mg is administered on the first, second [+/−one day], third [+/−one day], and fourth [+/−one day] days.

### BRIEF DESCRIPTION OF THE DRAWINGS

The foregoing will be apparent from the following more particular description of example embodiments of the invention, as illustrated in the accompanying drawings.

FIG. 1A and FIG. 1B: Molecular structure of primaquine and tafenoquine depicted together with summary biological data referenced in the text. Tafenoquine has a longer half-life in vivo and is consequently more potent with a broader spectrum of effects against multiple organisms.

FIG. 2: Desired structure of 8-aminoquinolines. R is any halogen-containing substituent of molecule weight 205.

FIG. 3: Survival curve for time to sustained clinical recovery in COVID-19 patients with tafenoquine vs. placebo.

FIG. 4A and FIG. 4B: Longitudinal effect of tafenoquine vs. placebo on MCP-1 in vaccinated vs. unvaccinated individuals with baseline MCP-1 levels <292 (FIG. 4A) or >292 (FIG. 4B). For FIG. 4A and FIG. 4B, MCP-1 levels at baseline are the actual recorded values and those at Day 5 are predicted values calculated from regression equations.

FIG. 5: In vitro antiviral SARS-CoV2 Data Report with tafenoquine succinate. Dose response curves and analytic data including IC50s [in microM] for remdesivir and tafenoquine against SARS-CoV-2 in CALU cells in vitro. Herein IC50 is used interchangeably with EC50. Calu3 cells were pretreated with test compounds prior to infection with SARS-CoV2 at an M01=0.5 for 48 hrs. Fixed cells were stained and imaged by microscopy for infection and cell number. Sample well data was normalized to aggregated DMSO control wells and plotted versus drug concentration to determine the IC50 (infection: blue) and CC50 (toxicity: green).

FIG. 6: In vitro antiviral SARS-CoV2 raw data report for tafenoquine in Calu3 cells.

FIG. 7A and FIG. 7B: Longitudinal effect of tafenoquine v placebo on MCP1 in patients with high or low baseline MCP 1 (<or >293 Units/nil) with BMI >25 (FIG. 7A) or <25 (FIG. 7B). MCP-1 levels at baseline are the actual recorded values and those at Days 5 are predicted values calculated from regression equations.

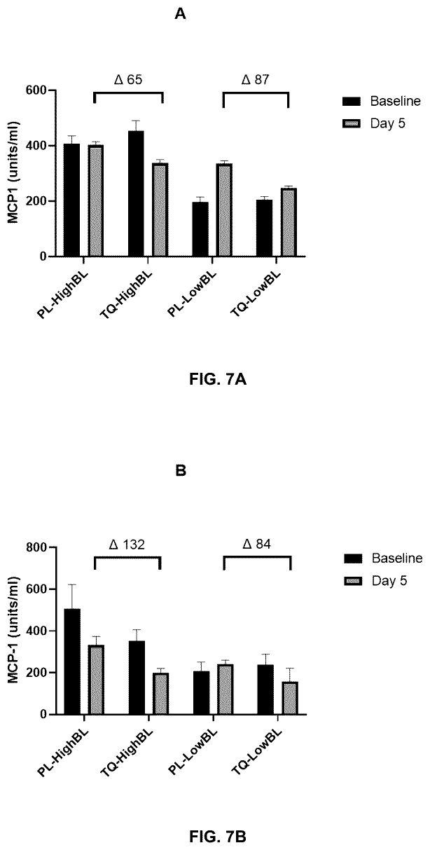

### DETAILED DESCRIPTION OF THE INVENTION

### Definitions

All definitions of substituents set forth below are further applicable to the use of the term in conjunction with another substituent. Unless defined otherwise, all technical and scientific terms used herein have the same meaning as commonly understood by one of ordinary skill in the art to which this invention belongs.

As used herein, the singular forms “a,” “and,” and “the” include plural reference unless the context clearly dictates otherwise. Additionally, the term “comprises” is intended to include embodiments where the method, apparatus, composition, etc., consists essentially of and/or consists of the listed steps, components, etc. Similarly, the term “consists essentially of” is intended to include embodiments where the method, apparatus, composition, etc., consists of the listed steps, components, etc.

As used herein, the term “about” refers to a number that differs from the given number by less than 15%. In other embodiments, the term “about” indicates that the number differs from the given number by less than 14%, 13%, 12%, 11%, 10%, 9%, 8%, 7%, 6%, 5%, 4%, 3%, 2%, or 1%.

As used herein, “asymptomatic” refers to a human subject that may or may not a have a disease or infection with MCP-1 involvement. In some embodiments, a subject that is asymptomatic for a given disease or infection has contracted the disease with MCP-1 involvement but is not currently showing symptoms resulting from that disease or infection. For example, an asymptomatic human subject includes a human subject that has no symptoms related to a SARS-CoV-2 infection and has been in close contact with someone that is infected with SARS-CoV-2. In another example, an asymptomatic human subject includes a human subject that has no symptoms related to a SARS-CoV-2 infection and has tested positive for infection of SARS-CoV-2.

As used herein, “G6PD” means Glucose-6-phosphate dehydrogenase and “G6PD deficiency” refers to a subject being deficient in this enzyme. In humans, treatment of a subject who has G6PD deficiency with an 8-aminoquinoline may cause hemolysis, which can be clinically significant in some cases.

As used herein, “G6PD-normal” refers to human subjects with normal levels of glucose-6-phosphate dehydrogenase. Normal levels of G6PD may be determined by approved laboratory tests using validated methodology known to those skilled in the art.

The human subject may be an adult or a child. As used herein, a “child” refers to a human subject who is between the ages of 1 day to 17 years of age. The term “adult” refers to a human subject who is 18 years of age or older.

As used herein, “loading phase” or “loading dose(s)” or “initial dose(s)” refers to the initial administration of the material and is at least one dose. For example, the loading phase may be once per day for three consecutive days or less prior to administration of less frequent administration of doses.

As used herein, “subsequent dose(s)” refers to doses administered after initial dose(s) and is at least one dose. The subsequent dose(s) may be the same amount or may be a different amount than the initial dose(s). The subsequent dose(s) may be administered in the same time frame or may be administered in a different time frame than the initial dose(s).

As used herein, “per day” means in a given 24-hour period.

As used herein, “per week” means in a given 7-day period.

“Three times a day dosing” or “three times per day,” as used herein, refers to three administrations of a composition per every 24-hour period.

“Four times a day dosing” (QDS) or “four times per day,” as used herein, refers to four administrations of a composition per every 24-hour period.

In particular, embodiments of the methods and compositions may use a pharmaceutical composition comprising a compound of Formula (I), or a pharmaceutically acceptable salt thereof. The disclosed compounds of Formula (I), or a pharmaceutically available salt thereof, can be administered to the subject in conjunction with an acceptable pharmaceutical carrier or diluent as part of a pharmaceutical composition for the methods described herein, and according to any of the dosing regimens described herein. Formulation of the compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

“Pharmaceutically acceptable carrier” means non-therapeutic components that are of sufficient purity and quality for use in the formulation of a composition of the invention that, when appropriately administered, typically do not produce an adverse reaction, and that are used as a vehicle for a drug substance (e.g., a compound of Formula (I), such as tafenoquine).

The phrase “pharmaceutically acceptable” indicates that the substance or composition must be compatible chemically and/or toxicologically, with the other ingredients comprising a formulation, and/or the mammal being treated therewith.

Pharmaceutical formulations include “pharmaceutically acceptable” and “physiologically acceptable” carriers, diluents or excipients. In this context, the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions.

The compounds of the present invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The phrase “effective amount” means an amount of an agent, such as an alpha-glucosidase inhibitor, that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, a subject is “in need of” a treatment if such human or non-human animal subject would benefit biologically, medically or in quality of life from such treatment (preferably, a human).

As used herein, the term “inhibit”, “inhibition” or “inhibiting” refers to the reduction or suppression of a given condition, symptom, or disorder, or disease (e.g., coronavirus infection, or coronavirus viral load or titer), or a significant decrease in the baseline activity of a biological activity or process (e.g., alpha-glucosidase production, inhibitors of glycoprotein processing, and inhibitors of alpha-glucosidase activity such as alpha-glucosidase I activity).

As used herein, a subject is “in need of” a treatment if such human subject would benefit biologically, medically or in quality of life from such treatment (preferably, a human). In some embodiments, the subject has a disease or infection with MCP-1 involvement and is in need of therapy. In other embodiments, the subject does not have a disease or infection with MCP-1 involvement and is in need of prophylaxis. In some embodiments, the subject in need of prophylaxis is at risk of having a disease or infection with MCP-1 involvement. In some embodiments, the subject is at increased risk of having a disease or infection with MCP-1 involvement relative to others in the population, or is at risk of having increased severity of a disease or infection ith MCP-1 involvement relative to others in the population.

As used herein, the terms “subject”, “patient”, and “individual” are used interchangeably and refer to a human of any age or gender.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset or development or progression of the disease or disorder).

As used herein, the term “pre-treat”, “pre-treating”, or “pre-treatment” of any disease or disorder refers to the embodiments described for “treat”, “treating”, or “treatment”, wherein the first administration is prior to having said disease or disorder. In particular embodiments, “pre-treat”, “pre-treating”, or “pre-treatment” of any disease or disorder refers to decreasing the severity of said disease or disorder or accelerating the recovery of said disease or disorder, wherein the first administration is prior to having said disease or disorder.

As used herein, the term “administration” is intended to include, but is not limited to, the following delivery methods: topical, oral, sub-lingual, buccal, parenteral, subcutaneous, transdermal, transbuccal, intravascular (e.g., intravenous or intra-arterial), intramuscular, subcutaneous, intranasal, and intra-ocular administration. Administration can be local at a particular anatomical site, such as a site of infection, or systemic.

As used herein, “prevent” or “prevention” refers to achieving, partially, substantially, or completely, one or more of the following results: avoiding a disease or infection with MCP-1 involvement; avoiding clinical symptom or indicator associated with a disease or infection with MCP-1 involvement; reducing the severity of a disease or infection with MCP-1 involvement; or avoiding a disease or infection with MCP-1 involvement.

The terms “strain” and “variant” are used interchangeably herein to refer to subtypes of a microorganism (e.g., a virus, bacterium, or fungus) that are genetically distinct from each other. For example, SARS-CoV-2 has multiple variants currently circulating globally. Such SARS-CoV-2 variants include at least Alpha (B.1.1.7 and Q lineages) identified in the United Kingdom, Beta (B.1.351 and descendent lineages) identified in South Africa, Gamma (P.1 and descendent lineages) identified in travelers from Brazil, Epsilon (B.1.427 and B.1.429), Eta (B.1.525), Iota (B.1.526), Kappa (B.1.617.1), 1.617.3), Mu (B.1.621 and B.1.621.1), Zeta (P.2), Delta (B.1.617.2 and AY lineages), and Omicron (B.1.1.529 and BA lineages). For example, SARS-CoV-2 variants may include mutations, such as the following: E484K, which was first discovered in the United Kingdom; L452R, which was detected in Denmark; and D614G discovered in China in January 2020. Other mutations identified in SARS-CoV-2 variants include, for example, the 69/70 deletion, 144Y deletion, N501Y, A570D, P681H, E484K, K417N/T, A67V, de169-70, T951, G142D, de1143-145, T547K, D614G, H655Y, N679K, D796Y, N856K, Q954H, N969K, L981F, delY144, T478K, G339D, S373P, S375F, N440K, S477N, E484A, Q493R, Q498R, Y505H, N764K, G142D, and S371L.

As used herein, “symptomatic” refers to a subject in whom symptoms of a disease or infection with MCP-1 involvement is evident upon clinical evaluation.

As used herein, “Coronavirus disease 2019” or “COVID-19” or “2019-nCoV acute respiratory disease” refers to the infectious disease caused by “severe acute respiratory syndrome coronavirus 2” also known as “SARS-CoV-2” or “Wuhan virus”.

As used herein, “tafenoquine” refers to a compound of Formula (I) with the following structure:

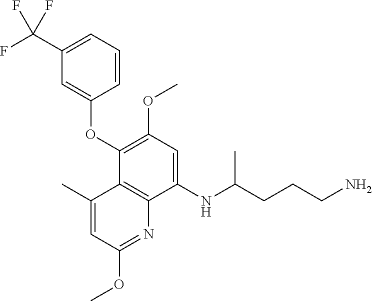 [SMILES: COc1cc(C)c2c(Oc3cccc(C(F)(F)F)c3)c(OC)cc(NC(C)CCCN)c2n1]

which has an alternative name of N(4)42,6-Dimethoxy-4-methyl-5-[3-(trifluoromethyl)phenoxy]quinolin-8-yl]pentane-1,4-diamine, or a pharmaceutical acceptable salt thereof. Tafenoquine may also be known as Tafenoquine [INN:BAN], Etaquine, UNII-262P8GS9L9, C24H28F3N303, CHEBI:172505, AIDS006901, 106635-81-8 (maleate), AIDS-006901, CID115358, SB-252263, WR 238605, WR-238605, WR238605, LS-172012, 1,4-Pentanediamine, N4-(2,6-dimethoxy-4-methyl-5-(3-(trifuluromethyl)phenoxy)-8-quinolinyl-, 106635-80-7, N(4)-(2,6-Dimethoxy-4-methyl-5-((3-trifluromethyle)phenoxy)-8-quinolinyI)-1,4-pentanediamine, N-[2,6-dimethoxy-4-methyl-5-[3-(trifluoromethyl)phenoxy]quinolin-8-yl]diamine, (4-Amino-1-methylbutyI){2,6-dimethoxy-4-methyl-5-[3-(trifluoromethyl)phenoxy](8-quinoly)}amine, (R)-N3-(2,6-Dimethoxy-4-methyl-5-(3-trifluoromethyl)phenoxy)quinolin-8-yl)pentane-1,4-diamine, (RS)-N(sup 3)-(2,6-Dimethoxy-4-methyl-5-(3-trifluoro-methylphenoxy)quinolin-8-yl)pentane-1,4-diamine. A pharmaceutically acceptable salt thereof, including,

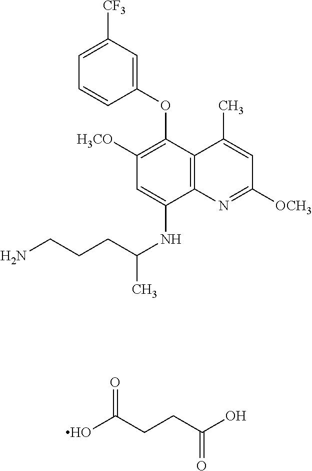

CAS number for above identified structure of succinate salt 106635-81-8. The compounds of the invention useful for practicing the methods described herein may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the invention.

The compounds described herein may exist in one or more tautomeric forms. All tautomers and mixtures thereof are included in the scope of the present invention. The compounds of the present invention can be administered as the free base or as a pharmaceutically acceptable salt. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estotate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts. In one embodiment, the compound of Formula (I) is a hydrochloride salt.

When used herein, a dose range reflected as two numbers means those doses as well as all doses within that range. For example, a dose range from 10 mg-11 mg means 10.0 mg, 10.05 mg, 10.10 mg, 10.15 mg, 10.20 mg, 10.25 mg, 10.30 mg, 10.35 mg, 10.40 mg, 10.45 mg, 10.50 mg, 10.55 mg, 10.60 mg, 10.65 mg, 10.70 mg, 10.75 mg, 10.80 mg, 10.85 mg, 10.90 mg, 10.95 mg, 11.00 mg, as well as any and all amounts therein, such as 10.34 mg, 10.78 mg, etc.

As used herein, “suspected COVID-19 disease” means a subject that has either been confirmed or not confirmed with a laboratory test for COVID-19 or who has been exposed to an individual with known or suspected COVID-19 disease.

As used herein, “a disease or infection with MCP-1 involvement” means a disease or infection wherein MCP-1 is upregulated as a result of or during the course of the disease or infection. Examples of diseases or infections with MCP-1 involvement include, but are not limited to COVID-19; influenza tuberculosis; gastric ulcer and bowel disease; diabetes; cancers, including breast carcinoma, triple negative breast cancer, and eosophageal squamous cell carcinoma; non-viral respiratory tract infections including tuberculosis; brain disorders including Alzheimer's disease, Parkinson's disease, and multiple sclerosis; osteoarthririts; rheumatoid arthritis; osteoporosis; diseases related to endothelial cell dysfunction; lupus nephritis; Lyme disease; fibromyalgia; and chronic fatigue syndrome.

### Long Half-Life 8-Aminoquinolines

Tafenoquine and other long half-life 8-aminoquinolines offer a therapeutic and prophylactic alternative to the standard of care for a disease or infection with MCP-1 involvement due to, in part, an exceptionally long half-life that broadens their modes of action relative to short half-life 8-aminoquinolines such as primaquine and the lack of demonstrated QTC prolongation in humans at therapeutically relevant doses.

8-aminoquinolines are known to target quiescent organisms such as the hypnozoites of P. vivax and the gametocytes of all malaria parasites [Llanos-Cuentos et al. 2019. N Engl J Med. 2019 Jan. 17; 380(3):229-241]. While many other anti-malarials, including tafenoquine and primaquine, target actively-dividing blood stages of malaria, only 8-aminoquinolines target the quiescent hypnozoites [Burrows et al. 2014. Parasitology. 2014 January;141(1):128-39. Epub 2013 July 17]. The mechanism of action of 8-aminoquinolines is thought to be via site specific activation to oxidative intermediates [Camarda et al 2019. Antimalarial activity of primaquine operates via a two-step biochemical relay. Nat Commun 10:3226 (Camarda et al 2019)]. Tafenoquine and other 8-aminoquinolines are active against both the quiescent and actively-dividing forms of Mycobacterium as well as other micro-organisms (with both replicating and non-replicating forms) associated with disease in humans and animals.

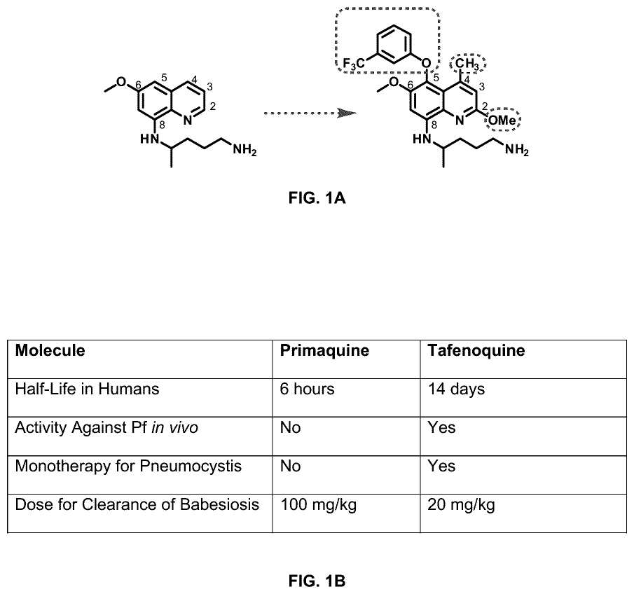

Extension of the half-life of 8-aminoquinolines through substitution at the 2, 4, and 5 positions of the quinoline ring is known to increase the potency and expand the spectrum of action compared to short half-life 8-aminoquinolines as primaquine. See FIG. 1A and FIG. 1B. For example, whereas primaquine has only week activity against the blood stages of P. falciparum, tafenoquine is quite effective [Baird et al. 2002. Am J Trop Med Hyg. 2002 June; 66(6):659-60; McCarthy et al. 2019. Clin Infect Dis. 2019 Jul. 18; 69(3):480-486]. Similarly, whilst tafenoquine can be administered as monotherapy to cure Pneumocystis infections in mice, primaquine must be combined with clindamycin to achieve the same outcome [Bartlett et al. ANTIMICROBIAL AGENTS AND CHEMOTHERAPY, February 1991, Vol 35(2): 277-282 (Bartlett et al. 1991)]. Also, whereas single doses of 20 mg/kg tafenoquine cleared Babesia parasitemia in mice [Mordue and Wormser 2019. 442 jid 2019:220, 1 August], a much higher dose of primaquine is required [100 mg/kg, Yao et al. 2015. J Infect Dev Ctries. 2015 Sep. 27; 9(9):1004-10].

Chloroquine and the related drug hydroxychloroquine have been proposed for both prevention and treatment of SARS-CoV-2, based on the well characterized mechanism of host cell lysosomal protonation, in vitro activity against SARS-CoV-2, accumulation in the lungs [the presumed site of viral replication], and the well characterized safety profile over sixty or more years of use to treat and prevent malaria and inflammatory conditions [Yao et al., In Vitro Antiviral Activity and Projection of Optimized Dosing Design of Hydroxychloroquine for the Treatment of Severe Acute Respiratory Syndrome Coronavirus 2 (SARS-CoV-2). Clin Infect Dis. 2020 March 9]. Data from two randomized studies and one large hospital registry study are available from hospitalized patients: The first of the these studies showed no difference in viral clearance compared to standard of care [Tang et al. BMJ May 14 2020], the second showed no difference in requirement for ICU transfer in patients requiring oxygen [Mahevas et al BMJ May 14 2020], and the third showed a higher rate of all-cause mortality including fatal cardiac arrythmias relative to the standard of care. Although these studies are not blinded, randomized, placebo-controlled investigations they have collectivity called into the question whether hydroxychloroquine is safe and effective in the general hospitalized COVID-19 population. Clinical trials were underway to evaluate the utility of chloroquine and hydroxychloroquine for prevention and treatment in an out-patient setting, but no results were available.

That hydroxychloroquine would cause cardiotoxicity in some individuals is not surprising given the high, daily doses administered for COVID-19 and the known propensity for this agent to prolong QTC interval, a major risk factor for drug-induced fatal arrythmias [Chorin et al 2020 Heart Rhythm May 11, online ahead of print]. The substantial increase in such events relative to the standard of care in COVID-19 patients [Mehra et al., Lancet May 22 2020, online ahead of print] is perhaps surprising, and may be because this patient population has a high incidence of comorbidities at baseline that also increase the risk of mortality and cardiac complications in COVID-19 disease. In any case, it would be beneficial to treat and prevent COVID-19 using therapeutic agents that are not cardiotoxic.

In a thorough QTC study, tafenoquine at doses of up to 1200 mg over three days were not found to increase the upper limit of 90% confidence interval of the QTC interval, thereby meeting the generally accepted regulatory standard for considering a drug to not exhibit a cardiotoxicity liability [Green et al 2014. J Clin Pharmacol 54:995-1005 (Green et al 2014)]. Importantly, tafenoquine also did not increase the QTC prolongation known to be associated with chloroquine when the two drugs were coadministered [Green et al 2014].

### Dosing Regimens

Dosing regimens according to the invention are those that are effective in preventing and/or treating a disease or infection with MCP-1 involvement in a given subject. Oral administration and/or formulation are done so as to minimize gastrointestinal (“GI”) upset in the subject, especially when doses 400 mg/day are given. Doses above 400 mg of tafenoquine are often not well tolerated (e.g., the dose may cause gastrointestinal issues or toxicity) by adult subjects regardless of the subjects' G6PD status. In G6PD normal adult subjects, doses of up to 400 mg of tafenoquine may be well tolerated, while in G6PD deficient subjects, doses of 300 mg or more may not be well tolerated. GI upset may be minimized and/or obviated and/or alleviated by buccal administration, sublingual administration, intravascular (e.g., intravenous or intra-arterial) administration, and/or by using a delivery design (tablet, sheet, etc.) that minimizes GI upset. Dosing may continue as necessary for up to six months provided the total dose administered does not exceed 10,800 mg in that six-month period.

In particular embodiments, the long half-life 8-aminoquinoline is tafenoquine, or a pharmaceutically acceptable salt thereof. The long half-life 8-aminoquinoline may be administered to the human subject as at least one initial (loading) dose. In particular embodiments, 100 mg-600 mg dose(s) are administered.

In further embodiments, the methods of treatment of a disease or infection with MCP-1 involvement and/or methods of prevention of a disease or infection with MCP-1 involvement, further comprise administering a second agent, such as a drug, to the human subject. In other embodiments, the method comprises administering to said subject an effective amount of a second agent. In further embodiments, the administration of tafenoquine or said compound of Formula (I) and administration of said second agent is concurrent. In yet other embodiments, the administration of tafenoquine or said compound of Formula (I) and administration of said second agent is not concurrent. In even further embodiments, no second agent is administered.

In other embodiments, the second agent is selected from the group consisting of amikacin, an aminoglycoside, amoxicillin, amphotericin formulations, any drug approved by the Food and Drug Administration for treating or preventing bacterial viral, and/or fungal infections, any azole-containing anti-fungal drug, atovaquone, azithromycin, Bactrim, bedaquiline, a benzothiazinone, BTZ043, capreomycin, cefftriaxime, cefotaxime, cefuroxamine, clindamycin, clofazimine, corticosteroids, a cyclic peptide, cycloserine, delamanid, a diarylquinoline, echinocandin, ethambutol, ethionamide, fluconazole, flucytosine, a fluoroquinolone, an imidazopyridine amide, isoniazid, itraconazole, kanamycin, levofloxacin, linezolid, a macrolide, moxifloxacin, a nitroimidazole, an oxazolidinone, PA-824, para-aminosolicyclic acid, PBTZ169, posaconazole, prothionamide, pyrazinamide, Q203, quinine, rifampin, rifapentine, SQ-109, streptomycin, sulfa drugs, sutezolid, a thioamide, trimethoprimsulfamethoxazole, vancomycin, voriconazole, any anti-viral drug, remdesivir, favipiravir, chloroquine, hydroxychloroquine, a monoclonal antibody treatment, a steroid, COVID-19 convalescent plasma, casirivimab, imdevimab, bamlanivimab, baricitinib, interleukin-6 inhibitors, kinase inhibitors, tyrosine kinsase inhibitors, Tocilizumab, ivermectin, and any combination thereof.

An embodiment of the invention is a dosing regimen according to Table 1 or 2, with or without a second agent. In particular, for either methods of treatment of a disease or infection with MCP-1 involvement or methods of prevention of a disease or infection with MCP-1 involvement, the method comprises administering to said subject an effective amount of tafenoquine or a compound of Formula (I), a pharmaceutically-acceptable salt thereof, or a pharmaceutical composition comprising tafenoquine or a compound of Formula (I),

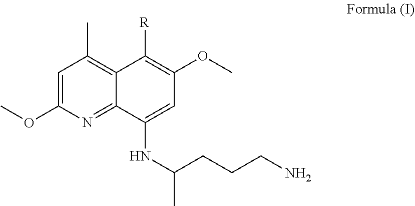

wherein R is any halogen-containing substituent of molecular weight 205, wherein the administering is in accordance with a dosing regimen according to Table 1. For example, such a dosing regimen may be used to treat or prevent a disease or infection with MCP-1 involvement, including, but not limited to, diseases or infections selected from the list of gastric ulcer and bowel disease, diabetes, cancers including breast carcinoma triple negative breast cancer, eosophageal squamous cell carcinoma, non-viral respiratory tract infections including tuberculosis, brain disorders including Alzheimer's disease, Parkinson's disease and multiple sclerosis, osteoarthririts, rheumatoid arthritis, osteoporosis, diseases related to endothelial cell dysfunction, lupus nephritisin, Lyme disease, fibromyalgia, and chronic fatigue syndrome symptomatic subjects with or without a second agent, using tafenoquine and/or long-half-life 8-aminoquinolines at the doses listed in Table 1, formulated appropriately. In another example, such a dosing regimen may be used to prevent and/or treat respiratory virus infections including SARS-CoV-2 with or without a second agent, using tafenoquine and/or long-half-life 8-aminiquinolines at the doses listed in Table 1 or 2, formulated appropriately.

TABLE 1

| Daily dose during | | | Weekly dose for the | | | | Additional |
|---|---|---|---|---|---|---|---|
| loading phase or | | | 4^{th}, 5^{th}, 6^{th} and 7^{th} | | | | doses if |
| during first three | | | doses of treatment (mg/ | | | | needed |
| days of treatment | | | week) if/as required | | | | (subsequent |
| (mg/day or mg/week) | | | (subsequent dose(s)) | | | | dose(s)) |
| Dose | Dose | Dose | Dose | Dose | Dose | Dose | Dose |
| 1 | 2 | 3 | 4 | 5 | 6 | 7 | 8+ |
| 400 | 400 | 400 | 400 | 400 | 400 | 400 | 400 |
| 440 | 440 | 440 | 440 | 440 | 440 | 440 | 440 |
| 480 | 480 | 480 | 480 | 480 | 480 | 480 | 480 |
| 520 | 520 | 520 | 520 | 520 | 520 | 520 | 520 |
| 560 | 560 | 560 | 560 | 560 | 560 | 560 | 560 |
| 600 | 600 | 600 | 600 | 600 | 600 | 600 | 600 |

TABLE 2

| | | | | Daily or weekly dose | | | | Additional |
|---|---|---|---|---|---|---|---|---|
| Daily dose during | | | | for the 4^{th}, 5^{th}, 6^{th} | | | | doses if |
| loading phase | | | | and 7^{th} doses of | | | | needed |
| or during first three or | | | | treatment (mg/day or | | | | (sub- |
| four days of treatment | | | | mg/week) if/as required, | | | | sequent |
| (mg/day or mg/week)* | | | | (subsequent dose(s))** | | | | dose(s))*** |
| Dose | Dose | Dose | Optional | Dose | Dose | Dose | Dose | Dose |
| 1 | 2 | 3 | Dose | 4 | 5 | 6 | 7 | 8+ |
| 100 | 100 | 100 | 100 | 100 | 100 | 100 | 100 | 100 |
| 130 | 130 | 130 | 130 | 130 | 130 | 130 | 130 | 130 |
| 160 | 160 | 160 | 160 | 160 | 160 | 160 | 160 | 160 |
| 190 | 190 | 190 | 190 | 190 | 190 | 190 | 190 | 190 |
| 220 | 220 | 220 | 220 | 220 | 220 | 220 | 220 | 220 |
| 250 | 250 | 250 | 250 | 250 | 250 | 250 | 250 | 250 |
| 280 | 280 | 280 | 280 | 280 | 280 | 280 | 280 | 280 |
| 310 | 310 | 310 | 310 | 310 | 310 | 310 | 310 | 310 |
| 340 | 340 | 340 | 340 | 340 | 340 | 340 | 340 | 340 |
| 370 | 370 | 370 | 370 | 370 | 370 | 370 | 370 | 370 |
| 400 | 400 | 400 | 400 | 400 | 400 | 400 | 400 | 400 |
| 440 | 440 | 440 | 440 | 440 | 440 | 440 | 440 | 440 |
| 480 | 480 | 480 | 480 | 480 | 480 | 480 | 480 | 480 |
| 520 | 520 | 520 | 520 | 520 | 520 | 520 | 520 | 520 |
| 560 | 560 | 560 | 560 | 560 | 560 | 560 | 560 | 560 |
| 600 | 600 | 600 | 600 | 600 | 600 | 600 | 600 | 600 |

*Doses ≤250 mg can be administered once per day for three or four days or once per week for three or four weeks. Doses >500 mg must be administered once per week. Doses 1-3 and optional dose do not need to be equal (e.g., Dose 1 may be 400 mg, Dose 2 may be 100 mg and Dose 3 may be 130 mg). Optional dose refers to a fourth dose administered during the loading phase or a dose given on the fourth day of treatment.
** Doses ≤250 mg can be administered once per day or once per week. Doses >250 mg must be administered once per week. In some embodiments, no subsequent doses are administered. In other embodiments, at least one subsequent dose is administered. Doses 4-7 do not need to be equal (e.g., Dose 4 may be 400 mg, Dose 5 may be 100 mg and Dose 6 may be 130 mg).
***Additional doses can be administered once daily until the cumulative dose reaches 1,600 mg and must be dosed weekly thereafter. Dose 8 does not need to be the same amount as Doses 8+ (e.g., Dose 8 may be 250 mg and Dose 9 may be 100 mg).

Diseases and Infections with MCP-1 involvement:

Monocyte chemotactic protein 1 (MCP-1) is a pro-inflammatory cytokine that is upregulated in many types of disease and infections and is often associated with the progression of severe disease and higher mortality rates. Non-clinical and clinical studies have demonstrated the role of upregulation of MCP-1 in the following disease or infection states: COVID-19, influenza tuberculosis, gastric ulcer and bowel disease, diabetes, cancers including breast carcinoma triple negative breast cancer, eosophageal squamous cell carcinoma, non-viral respiratory tract infections including tuberculosis, brain disorders including Alzheimer's disease, Parkinson's disease and multiple sclerosis, osteoarthririts, rheumatoid arthritis, osteoporosis, diseases related to endothelial cell dysfunction and lupus nephritis (Singh et al 2021). MCP-1 is also upregulated in vitro by Lyme disease spirachaetes and in animal models of Lyme disease, and down-regulation of MCP-1 and other pr-inflammatory cytokines is associated with clinical benefit (Parthasarathy et al 2013, Martinez et al 2015 and 2017). MCP-1 is upregulated in patients with fibromyalgia and chronic fatigue syndrome compared to healthy patients (Groven et al 2020).

In further embodiments said human subject, has one or more risk factors for disease progresses selected from the group consisting of: age of 60 years old or older, obesity, diabetes, and heart disease. In particular embodiments, said human subject has COVID-19 disease and has one or more risk factors for disease progresses selected from the group consisting of: age of 60 years old or older, obesity, diabetes, and heart disease.

The method may include detecting higher level of expression of MCP-1 compared to the expression level in a healthy subject or control sample, prior to administration of the long half-life 8-aminoquinoline. In other embodiments the method comprises determining the subject has a disease or infection with MCP-1 involvement and/or confirming elevated MCP-1 levels in the subject. Any suitable method for testing MCP-1 expression can be used, and such methods are well known in the art, including nucleic acid and protein assays. In some embodiments, said subject has been confirmed to have a disease or infection with MCP-1 involvement via laboratory test. Any suitable method for diagnosing such diseases or infections can be used, and such methods are well known in the art. In certain embodiments, the method involves diagnosing the subject as having a disease or infection that is known to have MCP-1 involvement without testing MCP-1 levels in the subject, while in other embodiments both disease diagnosis and MCP-1 expression are tested/evaluated. In other embodiments, said subject is clinically suspected to have a disease or infection with MCP-1 involvement.

In particular embodiments, the methods of treating a disease or infection with MCP-1 involvement and the methods of preventing a disease or infection with MCP-1 involvement, include methods of treating and methods of preventing symptoms thereof.

In some embodiments, administration of tafenoquine comprises a dosing regimen of 200 mg/day for three days following by 200 mg once weekly for as long as permitted by regulators. In other embodiments, administration of tafenoquine comprises the dose of tafenoquine as much as 399 mg at the same regimens as described herein, or multiple doses, such as up to 8 doses, as specified in Table 1 or Table 2.

In further embodiments, said human subject has at least one of the following conditions selected from the group consisting of: is at risk of catching respiratory virus during the winter season, [and therefore of contracting secondary infections], is elderly, is a surgical subject, has a catheter or iv line, has diabetes, has obesity, has COPD, has kidney disease, and has cardiac conditions. In further embodiments the subject is a child.

### EXEMPLIFIED EMBODIMENTS

Embodiment 1: A method for treating or preventing a disease or infection with MCP-1 involvement, or a symptom thereof, in a G6PD normal human subject, said method comprising administering an effective amount of a long half-life 8-aminoquinoline to a subject in need thereof.

Embodiment 2: The method according to embodiment 1, wherein the disease or infection is known to be associated with an increase in MCP-1 expression.

Embodiment 3: The method according to embodiment 1, wherein said human subject has a higher measured expression of MCP-1 compared to a person without said disease or infection.

Embodiment 4: The method according to any one of embodiments 1 to 3, further comprising administering a second agent.

Embodiment 5: The method according to any one of embodiments 1 to 4, wherein the method further involves testing level of MCP-1 in said subject prior to, during, and/or after administration of the long half-life 8-aminoquinoline.

Embodiment 6: The method according to any one of embodiments 1 to 5, wherein the long half-life 8-aminoquinoline is tafenoquine or a pharmaceutically acceptable salt thereof.

Embodiment 7: The method according to any one of embodiments 1 to 6, wherein the subject has the disease or infection with MCP-1 involvement at the time of said administering.

Embodiment 8: The method according to any one of embodiments 1 to 6, wherein the subject does not have the disease or infection with MCP-1 involvement at the time of administering, and wherein the long half-life 8-aminoquinoline is administered as prophylaxis.

Embodiment 9: A method for treating or preventing a disease or infection with MCP-1 upregulation, or a symptom thereof, in a human subject, said method comprising:
(a) administering to said subject an effective amount of a compound of Formula (I), a pharmaceutically-acceptable salt thereof, or a pharmaceutical composition comprising a compound of Formula (I), and

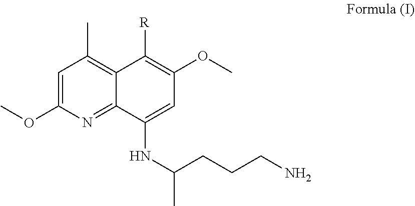

wherein R is any halogen-containing substituent of molecular weight 205, and wherein said lung infection is caused by at least one virus.

Embodiment 10: The method according to embodiment 9, further comprising determining said subject has said disease or infection with MCP-1 upregulation or is at risk of contracting said disease or infection with MCP-1 upregulation.

Embodiment 11: The method according to embodiment 9 or embodiment 10, further comprising administering a second agent.

Embodiment 12: The method according to any one of embodiments 9 to 11, wherein the subject is glucose-6-phosphate dehydrogenase (G6PD)-normal.

Embodiment 13: The method according to any one of embodiments 9 to 12, wherein said compound of Formula (I) is tafenoquine.

Embodiment 14: The method according to any one of embodiments 9 to 13, wherein the subject has the disease or infection with MCP-1 upregulation at the time of said administering.

Embodiment 15: The method according to any one of embodiments 9 to 13, wherein the subject does not have a disease or infection with MCP-1 upregulation at the time of administering, and wherein said compound of Formula (I) is administered as prophylaxis.

Embodiment 16: The method according to any one of embodiments 1 to 15, wherein at least seven doses of about 100 mg-600 mg are administered.

Embodiment 17: The method according to any one of embodiments 1 to 16, wherein said administration is via sub-lingual and/or buccal and/or intravenous route(s).

Embodiment 18: The method according to any one of embodiments 1 to 17, wherein said administration is conducted according to the dosing regimen of any one of Tables 1-2 and/or according to any of the Examples.

Embodiment 19: The method according to any one of embodiments 1 to 18, wherein no more than 10,800 mg is administered to said subject in a six-month period.

Embodiment 20: The method according to any one of embodiments 1 to 19, wherein about 100 mg to about 600 mg is administered in one or more initial dose(s).

Embodiment 21: The method according to any one of embodiments 1 to 20, wherein about 100 mg to about 600 mg is administered in one or more initial dose(s) and in one or more subsequent dose(s).

Embodiment 22: The method according to any one of embodiments 1 to 21, wherein three initial doses are administered once per day for three days.

Embodiment 23: The method according to any one of embodiments 1 to 22, wherein three or four initial doses are administered.

Embodiment 24: The method according to any one of embodiments 21 to 23, wherein the subsequence dose(s) is administered once per week.

Embodiment 25: The method according to any one of embodiments 21 to 23, wherein the subsequence dose(s) is administered once per day.

Embodiment 26: The method according to any one of embodiments 20 to 25, wherein the initial dose(s) is about 200 mg.

Embodiment 27: The method according to any one of embodiments 20 to 25, wherein the initial dose(s) is about 150 mg.

Embodiment 28: The method according to any one of embodiments 20 to 25, wherein the initial dose(s) is about 100 mg.

Embodiment 29: The method according to any one of embodiments 21 to 28, wherein the subsequent dose(s) is about 200 mg.

Embodiment 30: The method according to any one of embodiments 21 to 28, wherein the subsequent dose(s) is about 150 mg.

Embodiment 31: The method according to any one of embodiments 21 to 28, wherein the subsequent dose(s) is about 100 mg.

Embodiment 32: The method according to any one of embodiments 21 to 31, wherein the first subsequent dose is administered seven days after the last initial dose.

Embodiment 33: The method according to any one of embodiments 21 to 24, wherein an initial doses is about 200 mg and is administered once a day for three days, and wherein a subsequent dose is about 200 mg and is administered once a week.

Embodiment 34: The method according to any one of embodiments 21 to 33, wherein there is one subsequent dose administered approximately one week after the third initial dose.

Embodiment 35: The method according to any one of embodiments 20, 22, 23, 26, 27, and 28, wherein there are no subsequent doses.

Embodiment 36: The method according to any one of embodiments 1 to 35, wherein the disease or infection is gastric ulcer, bowel disease, diabetes, breast carcinoma, triple negative breast cancer, eosophageal squamous cell carcinoma, bacteria or fungi-associated respiratory tract infections, Alzheimer's disease, Parkinson's disease, multiple sclerosis, osteoarthririts, rheumatoid arthritis, osteoporosis, diseases related to endothelial cell dysfunction, lupus nephritis, COVID-19, SARS-CoV-2, a disease cause by SARS-CoV-2 with COVID-19-like symptoms persisting for more than 28 days, influenza, respiratory syncytial virus, Lyme disease, chronic fatigue syndrome and/or fibromyalgia.

Embodiment 37: A kit comprising: (a) a means for testing for G6PD deficiency; (b) a compound of Formula (I), a pharmaceutically-acceptable salt thereof, or a pharmaceutical composition comprising a compound of Formula (I); and

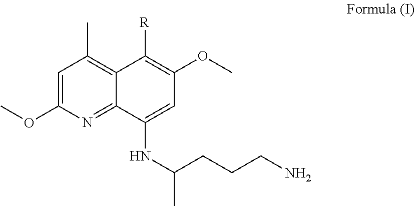

(c) instructions for use,
wherein R is any halogen-containing substituent of molecular weight 205.

Embodiment 38: The kit according to embodiment 37, further comprising (d) an effective amount of a second agent.

Embodiment 39: The kit according to embodiment 37 or embodiment 38, further comprising (e) means for testing for level of MCP-1 expression.

Embodiment 40: A method of treating a disease or infection in a human subject, the method comprising:
(a) determining that the human subject has said disease or said infection; and
(b) administering to said subject an effective amount of tafenoquine or a compound of Formula (I), a pharmaceutically-acceptable salt thereof, or a pharmaceutical composition comprising tafenoquine or a compound of Formula (I), and

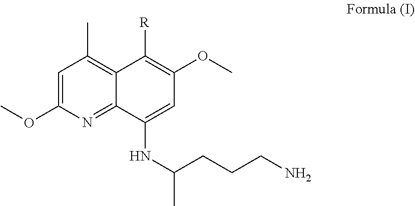

wherein R is any halogen-containing substituent of molecular weight 205, and wherein said disease or infection is an MCP-1 upregulated disease or infection.

Embodiment 41: The method according to embodiment 40, wherein said Formula (I) is tafenoquine or a salt thereof.

Embodiment 42: The method according to embodiment 40 or embodiment 41, wherein said effective amount comprises 600 mg administered over one to three days.

Embodiment 43: The method according to any one of embodiments 40-42, wherein said effective amount comprises 800 mg administered over one to four days.

Embodiment 44: The method according to any one of embodiments 40-43, wherein about 100 to about 800 mg of said compound of Formula (I) is administered in one or more initial dose(s) and in one or more subsequent dose(s).

Embodiment 45: The method according to any one of embodiments 40-44, wherein an initial dose of about 100 to 800 mg over one to four days is administered and then at least one dose of approximately 100 to 400 mg once weekly.

Embodiment 46: The method according to any one of embodiments 40-45, wherein at least one and up to 4 weekly doses of approximately 100 to 400 mg is administered following an initial dose of 100 to 800 mg.

Embodiment 47: A method of preventing a disease or infection in a human subject, the method comprising:
(a) determining that the human subject is at risk from said disease or infection; and
(b) administering to said subject an effective amount of tafenoquine or a compound of Formula (I), a pharmaceutically-acceptable salt thereof, or a pharmaceutical composition comprising tafenoquine or a compound of Formula (I), and

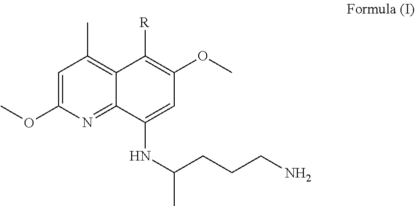

wherein R is any halogen-containing substituent of molecular weight 205, and wherein said disease or infection is an MCP-1 upregulated disease or infection.

Embodiment 48: The method according to embodiment 47, wherein said Formula (I) is tafenoquine or a salt thereof.

Embodiment 49: The method according to embodiment 47 or embodiment 48, wherein said effective amount comprises 600 mg administered over one to three days.

Embodiment 50: The method according to embodiment 47 or embodiment 48, wherein said effective amount comprises 800 mg administered over one to four days.

Embodiment 51: The method according to any one of embodiments 47-50, wherein about 100 to about 800 mg of said compound of Formula (I) is administered in one or more initial dose(s) and in one or more subsequent dose(s).

Embodiment 52: The method according to any one of embodiments 47-51, wherein an initial dose of about 100 to 800 mg over one to four days is administered and then at least one dose of approximately 100 to 400 mg is administered once weekly.

Embodiment 53: The method according to any one of embodiments 47-52 in which at least one and up to 51 weekly doses of approximately 100 to 400 mg is administered following an initial dose of 100 to 800 mg.

Embodiment 54. The method according to any one of embodiments 40-53, further comprising administering a second agent.

Embodiment 55: The method according to any one of embodiments 40-54, wherein the disease or infection is gastric ulcer, bowel disease, diabetes, breast carcinoma, triple negative breast cancer, eosophageal squamous cell carcinoma, bacteria or fungi-associated respiratory tract infections, Alzheimer's disease, Parkinson's disease, multiple sclerosis, osteoarthririts, rheumatoid arthritis, osteoporosis, diseases related to endothelial cell dysfunction and lupus nephritis, COVID-19, SARS-CoV-2, influenza, respiratory virus, Lyme disease, chronic fatigue syndrome and/or fibromyalgia.

Embodiment 56: The method according to any one of embodiments 1 to 35, where in the condition being treated are adverse events associated with m-RNA vaccination for COVID-19 or other diseases.

Embodiment 57: The method according to any one of embodiments 1 to 35 or 40 to 56, wherein said administering is conducted according to the dosing regimen of any one of Table 1 and/or Table 2 and/or according to any of the Examples.

### EXAMPLES

### Example 1—Substituted 8-Aminoquinolines are More Active and have Broader Spectrum of Activity than Primaquine

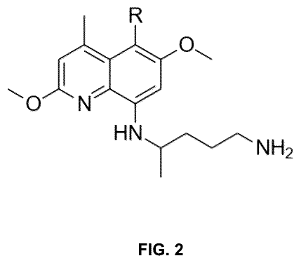

Tafenoquine is more potent and has a broader spectrum of activity against malaria parasites and Pneumocystis in vivo as a consequence of its longer half-life (14 days versus approximately 6 hours). This occurs as a consequence of substitutions at the 2, 4, and 5 positions that increase steric bulk, lipophilicity, and block sites of metabolic attack. Tafenoquine and similarly substituted 8-aminoquinolines such as those illustrated in FIG. 1A and FIG. 2 exhibit more potent and broader spectrums of action against lung pathogens and lung diseases of humans and animals in a manner similar to what is described in the examples for tafenoquine.

### Example 2—Approved Direct Inhibitors of Viral Replication Reduce Deaths but do not Accelerate Recovery from Mild-Moderate Symptoms of COVID-19 Disease

Nirmatrelvir and molnupiravir are approved small molecule therapeutics that directly inhibit SARS-CoV-2 replication in COVID-19 patients, manifest as a reduction in nasopharyngeal load as measured in clinical trials. Nirmatrelvir, In combination with ritonavir (a cytochrome P450 inhibitor) as “paxlovid”, and molunpiraivr, are reported by their manufacturers to be effective for reducing the risk of hospitalization and death when administered to patients with COVID-19 disease. However, paxlovid does not exhibit any utility for the acceleration of time to sustained clinical recovery (TTCR) from all COVID-19 symptoms, where sustained recovery refers to improvement in all COVID-19 symptoms for four days or more.

### Example 3—Tafenoquine Reduced Time to Clinical Recovery from Fever, Cough and Shortness of Breath in a Phase II Clinical Trial

Tafenoquine, administered as the first four 200 mg doses of the FDA-approved regimen for malaria prophylaxis (200 mg on Days 1, 2, 3 and 10) was shown in exploratory analyses to accelerate recovery from fever, cough and shortness of breath in patients recovering from mild-moderate COVID-19 disease (P<0.02, Dow and Smith 2022). Laboratory endpoint and patient-level individual symptom data were collected in that study, have not been reported elsewhere, and form the basis for surprising findings about the therapeutic and mechanistic effects of tafenoquine described herein.

### Example 4—Tafenoquine Surprisingly Accelerates Time to Sustained Clinical Recovery in COVID-19 Patients and Reduces the Risk of Persistent Symptoms at Day 28

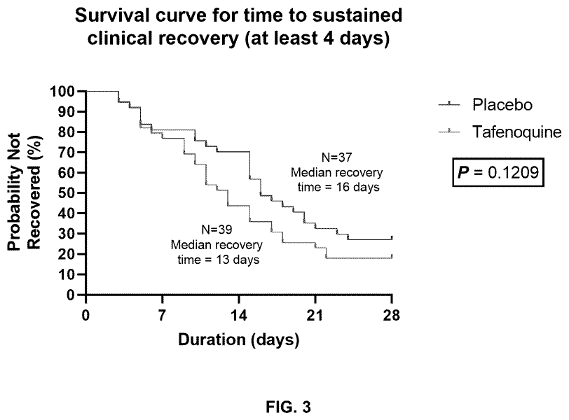

Longitudinal aggregate symptom data for individual patients from Dow and Smith (2022) were examined to determine the number of days required before each patients reported an aggregate symptom score of 2 for four days of more for the first time. This endpoint was not included as a pre-specified analysis in the original study design, and was only recently confirmed to be an endpoint of interest to regulars in mild-moderate COVID-19 disease in standard risk patients. Patients who were hospitalized or lost to follow up in the original study, or who did not have an aggregate symptom score 3 on the first day of drug administration were excluded from the analysis. A survival curve was generated, and data were analyzed using Graphpad Prism. Tafenoquine exhibited a median time to clinical recovery of 13 days whereas for placebo it was 16 days, and the curves separated after approximately one week. At Day 28, which was the last day of the study, the proportion of patients with persistent COVID-19 symptoms was lower in the tafenoquine arm. As shown in FIG. 3.

### Example 5—Tafenoquine Downregulates the Cytokine MCP-1 in COVID-19 Patients

Blood samples for a cytokine panel analysis were collected at Baseline, Day 5 and Day 14, and analyzed using standard commercially available tests from ACM Labs. Only samples with data from Baseline/Day 5 or Baseline/Day 14 were analyzed. Levels for interferon gamma (IFG), interleukin 10 (IL10), tumor necrosis factor (TNF), interleukin 1beta (IL1B), interleukin 12 (IL12), interleukin 2 (IL2), interleukin 4 (IL4), and interleukin 6 (I L6) were only above the detection limit in about 10% of patients, so these were not further analyzed. Monocyte chemotactic protein 1 (MCP-1) levels were detectable at baseline and Day 5 or 14 for all patients in which paired samples could be obtained. No adjustments needed to be made for limits of detection or quantification. Changes from baseline were calculated by subtracting the baseline value from the Day 5 or Day 14 value.

Multivariate regression analysis (performed utilizing the standard built-in program in Graphpad Prism 9.3.1) was used with the following dependent variables: MCP-1 change from baseline through Days 5 (Regression 1) and MCP-1 change from baseline through Day 14 for patients with baseline MCP-1 exceeding 350 units/ml (Regression 2) and less than 350 units/ml (Regression 3). The dependent variable data were pre-treated in the following manner to ensure that they passed at least three of four tests of normality (Anderson-Darling, D'Agostino-Pearson, Shapiro-Wilk and Kolmogorov-Smirnov tests) according to the standard built-in programs in GraphPad. One data point was removed from each of the MCP-1 datasets using Graph Pad's outlier removal function.

Treatment (tafenoquine=1, placebo=0) was included as an independent variable in all analyses. The following independent variables potentially related to COVID-19 disease outcomes were considered for inclusion if their univariate P-values were <0.25: Vaccination (yes or no), age (years), gender (male=1, female=0), body mass index (kg/m²), duration of symptoms prior to treatment with study medication (days), aggregate Day 1 symptom score, a risk factor assignment where age exceeded 64 (>64=1, ≤64=0), a risk factor assignment where BMI was exceed 25 (>25=1, ≤25=0), a risk factor assignment were BMI was >31 (>31=1, ≤31=0), and the baseline values for each dependent variable of interest. Where age, BMI, or a related risk factor were considered, only one of the related variables was included in the final model. For each dependent variable, regressions were performed iteratively to generate that with the strongest association with tafenoquine. That model for each dependent variable is presented in Table 3.

Temporal changes in MCP-1 were visualized using actual measured baseline values, and Day 5 values calculated using the relevant regression equations. Since actual values for Day 5 were not captured from all the patients with a baseline reading, this visualization approach allowed examination of trends from a larger patient pool.

Statistically significant regressions were obtained for all endpoints (Table 3), and r² for all regressions exceeded 0.27 in all cases. Tafenoquine was a statistically significant covariate only for MCP-1 change from baseline through Day 5 (P=0.0093, regression #1, Table 3). For all three regressions the baseline MCP-1 level was a significant covariate (Table 3). Additional regressions were performed for the original unedited datasets for MCP1 change from baseline and antibody change from baseline—these resulted in generally the same conclusions as presented in Table 3.

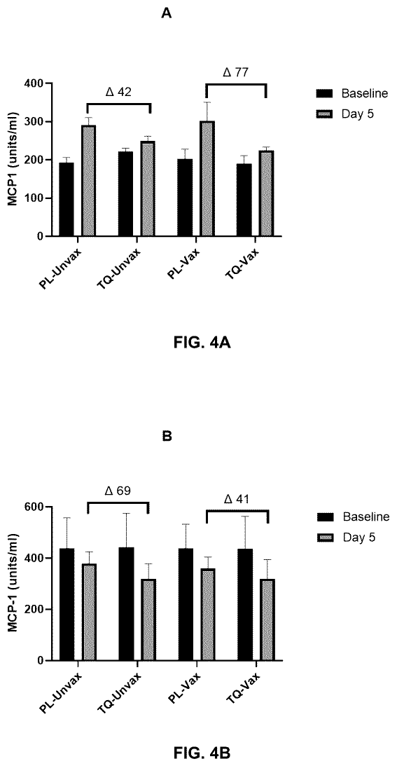

Changes in predicted MCP-1 levels from baseline through Day 5 were examined visually in a subgroup analysis involving three variables: treatment (tafenoquine or placebo), vaccination (vaccinated or unvaccinated) or baseline MCP-1 level (<or >median value which was =292). A high baseline level of MCP-1 (>292) was associated with declines in MCP-1 from baseline to Day 5 regardless of treatment group (FIG. 4A). In contrast a low baseline level of MCP-1 (<292) was associated with increases in MCP-1 from baseline to Day 5 regardless of treatment group (FIG. 4A). Tafenoquine was associated with a reduction in Day 5 MCP-1 of at least 41/10% in all group comparisons (FIG. 4A and FIG. 4B), independent of vaccination or baseline MCP-1 status.

Tafenoquine has never before been shown to downregulate MCP-1 in patients, in vitro or in animal models.

TABLE 3
Regression analysis of MCP-1 change from baseline

| | Regression | Regression | Regression | Regression | Re- |
|---|---|---|---|---|---|
| | 1: MCP1 | 2: MCP1 | 3: MCP1 | 1: | gression: |
| | Day 5 CFB | Day 14 CFB | Day 14 CFB | MCP1 | MCP1 |
| | (Multi- | for high baseline | for low baseline | Day 5* | Day 14 |
| | variate)* | MCP1* | MCP1* | (Univariate) | CFB*** |
| Outputs/Variables | Metrics for overall regression | | | | |
| Summary | | | | | |
| P | <0.0001 | <0.0065 | <0.045 | N/A | 0.0005 |
| r² | 0.60 | 0.57 | 0.27 | N/A | 0.32 |
| DF | 45 | 14 | 25 | 49 | 45 |
| Normality tests—residuals¹ | 1 | 4 | 4 | N/A | |
| Variable collinearity (r²)² | <0.16 | <0.03 | <0.05 | N/A | |
| | Metrics for individual variables | | | | |
| Variables | Beta/P | | | | |
| Intercept | 145/ | 421/ | 95.4/ | N/A | 33.3/ |
| | 0.0029 | 0.0456 | 0.0941 | | <0.0001 |
| Day 1 aggregate symptom score | N/A | N/A | N/A | N/A | N/A |
| Vaccination (vacc. = 1, unvacc, = 0) | N/A | 259/ | N/A | N/A | N/A |
| | | 0.0160 | | | |
| Treatment (TQ = 1, PL = 0) | −74.4/ | 78.9/ | −35.0/ | −45.2/ | 0.584// |
| | 0.0093 | 0.3304 | 0.2356 | 0.2548 | 0.872 |
| Symptoms prior to Day 1 (days) | N/A | N/A | N/A | N/A | N/A |
| Gender (Male = 1) | 30.5/ | N/A | N/A | N/A | N/A |
| | 0.2812 | | | | |
| BMI | N/A | N/A | N/A | N/A | N/A |
| Age (year) | N/A | N/A | N/A | N/A | N/A |
| Age risk factor (>64 = 1, ≤64 = 0) | −33.5/ | N/A | 144/ | N/A | 12.5/ |
| | 0.4182 | | 0.0137 | | 0.0228 |
| BMI risk factor (>25 = 1, ≤25 = 0) | 92.3/ | N/A | N/A | 65.2/ | N/A |
| | 0.0094 | | | 0.1869 | |
| BMI risk factor (>31 = 1, ≤31 = 0) | N/A | N/A | N/A | N/A | N/A |
| Baseline value (for lab endpoints) | −0.631/ | −1.35/ | −0.247/ | −0.674/ | −0.028/ |
| | <0.0001 | 0.0027 | 0.2430 | <0.0001 | 0.0002 |

*CFB = Day 5 value − Baseline value (i.e. a negative value means a decrease from baseline).
**CFB = Day 14 value − baseline value (i.e. a negative value means a decrease from baseline).
***Day 14 − BL change from baseline data for this variable were transformed such that the largest decline in MCP-1 from baseline through Day 14 was assigned a rank of 1. Beta for this variable is based on the transformed data—thus a positive coefficient implies an increase in the rank of the MCP1 change from baseline.
****CFB = Day 5 − Day 1 aggregate symptom score (i.e. a negative value represents an improved/symptom score).
¹Baseline variable was MCP1 baseline.
N/A means variable not used in multivariate analysis or not applicable to univariate analysis.
Statistically significant covariates are highlighted in bold.
¹Number of four different tests for normality for residuals which were passed.
²All independent variables had R² values for collinearity with other variable less than the number indicated.

In the univariate regressions at least one of the MCP1 variables exhibited a P value <0.25 for all aggregate symptom score variables except for Day 1 aggregate symptom score. Multivariate regression analyses in which an MCP1 independent variable was significantly correlated with a dependent aggregate symptom score variable are presented in Table 4 (regression 5 and 6). The aggregate symptom score at Day 5 (regression 5) was correlated with Day 1 aggregate symptom score (p=0.0071) and Day 5 MCP 1 (p=0.0194) and the overall regression was statistically significant (r²=0.23, p=0.0017). The change in aggregate symptom score from Day 1 through Day 5 (regression 6) was correlated with Day 1 aggregate symptom score (p=0.0003) and baseline MCP1 levels (P=0.0271) and the overall regression was statistically significant (r²=0.25, p=0.0002). Day 1 symptoms and the MCP1 endpoints were also correlated with clinical outcome in univariate regressions (see Table 4). The coefficients for the MCP1 variables for these regressions demonstrate that increased MCP1 at Day 5 is associated with increased aggregate symptom score at Day 5 (regression 5), and that a higher baseline MCP1-1 value is associated with less recovery from COVID-19 symptoms (regression 6).

TABLE 4
Regression analysis of Day 5 aggregate
symptom scores and change in aggregate
symptom score from Baseline through Day 5.

| | Regression | | Regression | |
|---|---|---|---|---|
| | 5: | Re- | 6: | Re- |
| | Day 5 | gression | D1 to D5 | gression |
| | Aggregate | 5: | Symptom | 6: |
| | Symptom | Univariate | Score | Univariate |
| | Score | Analyses | CFB* | Analyses |
| Outputs/Variables | Metrics for overall regression | | | |
| P | 0.0015 | N/A | 0.0002 | N/A |
| r² | 0.23 | N/A | 0.25 | N/A |
| DF | 49 | N/A | 60 | N/A |
| | Metrics for individual variables | | | |
| Variables | Beta/P | | | |
| Intercept | −1.27/ | N/A | −1.11/ | N/A |
| | 0.5974 | | 0.5268 | |
| Day 1 aggregate | 0.333/ | 0.3631/ | −0.458/ | −0.533/ |
| symptom score | 0.0071 | 0.0002 | 0.0003 | <0.0001 |
| MCP1 Day 5 | 0.013/ | 0.014/ | Not inc | Not inc |
| | 0.0194 | 0.0168 | | |
| Baseline value | Not inc | Not inc | 0.005/ | 0.005/ |
| | | | 0.0271¹ | 0.0457¹ |

*CFB = Day 5 − Day 1 aggregate symptom score (i.e. a negative value represents an improved/symptom score).
¹Baseline variable was MCP1 baseline.
N/A means variable not used in multivariate analysis or not applicable for univariate analyses.
Not included means that variable was excluded from the analysis.

### Example 6—Tafenoquine does not Reduce Nasopharyngeal Load or Alter Change in Antibody Levels from Baseline

Blood samples were collected at Baseline and Day 14 and total anti SARS-CoV-2 spike protein antibody was determined utilizing the standard commercially available assay from Eurofins. Only samples from patients where Baseline and Day 14 values were obtained were analyzed. Samples with values below the limit of detection were assigned a value of half the limit of detection. Raw data were log 2 transformed, and the fold-change from baseline was calculated by subtracting the log 2 baseline value from the log 2 Day 14 value. Log2 baseline and Log2 change from baseline values were used in the analyses. Data from patients who had the maximum quantifiable value at baseline (mostly but not exclusively vaccinated patients) were excluded from analysis.

NP swabs were collected at Baseline and Day 5 and Log10 viral copy number/ml was determined utilizing the standard commercially available assay from Eurofins. Only samples from patients where valid baseline and Day 5 results were obtained were analyzed. Samples with values below the dynamic range of the assay were assigned a value equivalent to the detection limit. Samples with detectable virus were assigned a value equivalent to half the detection limit. Viremia change from baseline was calculated by subtracting log 10 Day 5 copy number from the log 10 baseline copy number. Log10 Baseline and change from baseline values were used in the analyses.

Multivariate regression analysis (performed utilizing the standard built-in program in Graphpad Prism 9.3.1) was used to analyze the following dependent variables: Change in NP viral load from baseline through Day 5 (Regression 1) and total anti SARS-CoV-2 antibody change from baseline through Day 14 (Regression 2). The dependent variable data were tested to ensure that they passed at least three of four tests of normality (Anderson-Darling, D'Agostino-Pearson, Shapiro-Wilk and Kolmogorov-Smirnov tests) according to the standard built-in programs in GraphPad. The same independent variables described in Example 6 were utilized. Baseline viremia and log 2 baseline antibodies were also included as independent variables in the relevant regressions. The same iterative approach was utilized to generate the regression analyses with the strongest associated between tafenoquine and the dependent variables.

As seen from examining Table 5, treatment was not a statistically significant (P<0.05) covariate in either model.

TABLE 5
Regression analysis of nasopharyngeal viral load and
anti-SARS-COV-2 antibody changes from baseline

| | Regression 1: | Regression 2: |
|---|---|---|
| | NP Viral | Antibodies CFB |
| | Load CFB* | (unvaccinated)** |
| Outputs/Variables | Metrics for overall regression | |
| P | <0.0001 | <0.0001 |
| r² | 0.48 | 0.55 |
| DF | 58 | 39 |
| | Metrics for individual variables | |
| Variables | Beta/P | |
| Intercept | 3.33/ | 6.80/ |
| | 0.0338 | <0.0001 |
| Treatment (TQ = 1, PL = 0) | −0.0735/ | −0.671/ |
| | 0.8650 | 0.3367 |
| Symptoms prior to Day 1 (days) | −0.560/ | N/A |
| | 0.0052 | |
| Gender (Male = 1) | 1.05/ | N/A |
| | 0.0201 | |
| Age risk factor (>64 = 1, ≤64 = 0) | 2.67/ | N/A |
| | 0.0003 | |
| BMI risk factor (>25 = 1, ≤25 = 0) | N/A | N/A |
| Baseline value | −0.621/ | −0.492/ |
| | <0.0001 | <0.0001 |

*CFB = Day 5 value − Baseline value (i.e. a negative value means a decrease from baseline).
**CFB = Day 5 or Day 14 value − baseline value (i.e. a negative value means a decrease from baseline).
¹Baseline variable was MCP1 baseline.
N/A means variable not used in multivariate analysis or not applicable to univariate analysis.

### Example 7—MCP-1 is Upregulated and Associated with Disease Progression in SARS-CoV-2 and Influenza Infections

MCP-1 is a chemokine induced by a variety of tissue types under a variety of disease states in response and recruits monocytes and other immune cells to sites of inflammation and infection [Singh 2021]. MCP-1, through this and other mechanisms plays an important role in a number of disease states [Singh 2021]. In the acute phase of COVID-19 disease, MCP-1 is elevated relative to healthy patients, and high levels of circulating MCP-1 are correlated with disease progression and higher rates of hospitalization and mortality [Teixeira et al 2021, Marques et al 2022, Mulla et al 2022, Kumbeyono et al 2022]. MCP-1 is also elevated in other respiratory virus infections, such as influenza, relative to healthy patients [Betacova 2017, Sledkpva 2006]. When used to treat or prevent viral diseases caused by SARS-CoV-2, influenza, or other viruses where MCP-1 is upregulated, tafenoquine is expected to accelerate recovery in moderate disease and lower the risk of disease progression, hospitalization and death. The syndrome of “long-COVID”, which is caused by dysregulation of the immune system following an infection by SARS-CoV-2, and in which COVI-19-like (and other) symptoms present in the acute phase persist beyond 28 days, is still being studied, but would be expected to have MCP-1 involvement. Tafenoquine is also expected to provide benefit in this population.

### Example 8—MCP1 is Upregulated by mRNA Vaccination, and Adverse Events Associated with Vaccination are Expected to be Mitigated by Tafenoquine

In an animal model, MCP-1 was found to be upregulated by mRNA based SARS-CoV-2 spike protein and HIV vaccines to a substantially greater degree than DNA and protein vaccines (Makda et al 2022). In example 6, vaccination was associated with elevated MCP-1 (P<0.02, Table 3), and in example 7 tafenoquine did not affect total anti-SARS-CoV-2 antibody generation (P=0.546, Table 4). Thus, is is expected that tafenoquine would reduce mRNA vaccine associated MCP-1 increases and relieve associated symptoms without affecting vaccine-induced production of antibodies.

### Example 9—Tafenoquine is Expected to Provide Clinical Benefit in Other Disease States where MCP-1 is Upregulated

Non-clinical and clinical studies have demonstrated the role of upregulation of MCP-1 in the following disease states: gastric ulcer and bowel disease, diabetes, cancers including breast carcinoma triple negative breast cancer, eosophageal squamous cell carcinoma, non-viral respiratory tract infections including tuberculosis, brain disorders including Alzheimer's disease, Parkinson's disease and multiple sclerosis, osteoarthririts, rheumatoid arthritis, osteoporosis, diseases related to endothelial cell dysfunction and lupus nephritis (Singh et al 2021). MCP-1 is also upregulated in vitro by Lyme disease spirachaetes and in animal models of Lyme disease, and down-regulation of MCP-1 and other pro-inflammatory cytokines is associated with clinical benefit (Parthasarathy et al 2013, Martinez et al 2015 and 2017). MCP-1 is upregulated in patients with fibromyalgia and chronic fatigue syndrome compared to healthy patients (Groven et al 2020). Tafenoquine is expected to have benefit in these disease states through down-regulation of MCP-1 and other inflammatory processes when used alone or in combination with standard of care therapies for treatment of these conditions.

### Example 10—Tafenoquine Inhibits SARS-CoV-2 Replication In Vitro

In vitro susceptibility of viruses to an antiviral agent may be assessed using a quantitative assay to measure virus replication in the presence of increasing concentrations of the product compared to replication in the absence of the product. The effective concentration is the concentration of product at which virus replication is inhibited by 50 percent (EC50 for cell-based assays). Assays that evaluate antiviral activity include, but are not limited to, virus inactivation assays, plaque reduction assays, cytopathic effect inhibition assays, peripheral blood mononuclear cell (PBMC) assays, and binding and fusion assays [FDA Guidance for Industry, Antiviral Product Development—Conducting and Submitting Virology Studies to the Agency. U.S. Department of Health and Human Services, Food and Drug Administration, Center for Drug Evaluation and Research (CDER). June 2006].

### 4 Day Assay:

The in vitro susceptibility of SARS-CoV-2 to tafenoquine was first assessed in a 4-day cytopathic effect (“CPE”) inhibition assay and then is being assessed in a 2-day TCID-50 assay.

Briefly, for the 4-day CPE assay, Vero E6 cells were seeded into 96-well plates at 2x10⁴ cells/well in 100 μL seeding media (Minimal Essential Medium supplemented with 1% (w/v) L-glutamine, 2% fetal bovine serum). Plates were incubated overnight at 37° C., 5% CO₂.

A 9-point, 3-fold dilution series was initially prepared in DMSO (25,000 uM-3.8 uM) followed by transfer of a volume of each compound dilution into virus growth media (Minimal Essential Medium supplemented with 1% (w/v) L-glutamine, 2% FBS, 4 μg/mL TPCK-Trypsin). Each tafenoquine intermediate dilution series was added to the pre-seeded Vero E6 plates so that the final concentration range was 50 uM-7.6 nM.

SARS-CoV-2 diluted in virus growth media to generate a moi of 0.05, was added to the 96-well plates. This moi was previously determined to provide 100% CPE in 4 days. Virus was added to triplicate rows to assess viral activity and virus growth media without virus was added to triplicate rows to assess cytotoxicity. Plates were incubated at 37° C., 5% CO₂ for 4 days prior to staining with MTT.

After incubation for four days, viable cells were determined by staining with MTT. A solution of MTT was added to plates (final concentration 1 mg/mL) and incubated for 2 hours at 37° C. in a 5% CO₂ incubator. Wells were aspirated to dryness and formazan crystals solubilised by the addition of 2-Propanol. Absorbance was measured at 540-650 nm on a plate reader. The percent cell protection achieved by the positive control and test articles in virus-infected cells was calculated by the formula of Pauwels et al Rapid and automated tetrazolium-based colorimetric assay for the detection of anti-HIV compounds J VI ROL METHODS 1988 August;20(4):309-21 and the EC50 values were calculated via non-linear regression.

The 50% cytotoxic concentration (CC50) was defined as the concentration of the test compound that reduced the absorbance of the mock infected cells by 50% of the control value.

Remdesivir and hydroxychloroquine were used as positive controls.

Tafenoquine exhibited an EC50 of 15.7 microM, a CC50 of 37.2 microM, with a selectivity index of 2.4. Remdesivir exhibited at EC50 of 0.7 uM, a CC50 of >100 uM, and a selectivity index of >143 uM. Hydroxychloroquine exhibited 43% inhibition at the highest non-toxic concentration [33 microM], meaning that an EC50 could not be calculated. The CC50 of hydroxychloroquine was approximately 55 microM.

The difference in EC50 between remdesivir and tafenoquine may be because remdesivir is a direct antiviral, whereas tafenoquine alters host cell physiology that offers a mechanism of viral replication different or complementary to other quinolines.

48 hour assay:

The reduction in virus titre after exposure to tafenoquine for a 48-hour period was assessed via Tissue Culture Infective Dose 50 (“TCID50”).

A 5-point, 3-fold dilution series of Tafenoquine (50 uM-0.6 uM) was prepared in assay media and added to Vero E6 cells, pre-seeded overnight in 24 well plates. SARS-CoV-2 was diluted in virus growth media to generate a moi of 0.05 and was added to the 24-well plates and plates incubated for 48 hours. The remaining virus was quantified via TCI D50 assay. Plates were incubated for three days at 37° C. in a humidified 5% CO₂ atmosphere, and virus-induced CPE scored visually. The TCID50 of the virus suspension was determined using the method of Reed L J, Muench H. A simple method of estimating fifty percent endpoints. Am J Hyg. 1938; 27:493-7.

Hydroxychloroquine was used as a positive control.

Tafenoquine exhibited an EC50 of 2.6 microM. The selectivity index, calculated relative to the CC50 of 37.2 microM for the 4-day test, was 14.3. Hydroxychloroquine exhibited an EC50 of 10.4 microM. The selectivity index, calculated relative to the CC50 of 67 microM for the 4-day test, was 5.3.

The increased potency and selectivity of tafenoquine in the 48h assay compared to the 96h assay is presumably because the number of replication cycles in the 48h assay are fewer.

These data demonstrate that tafenoquine has intrinsic activity against SARS-CoV-2 that may provide clinical benefit, and exhibits much greater potency and selectivity than hydroxychloroquine.

### Example 11: Tafenoquine Exhibits Surprising Antiviral Activity Against SARS-CoV-2 in Human Respiratory Cells and is More Active than Other Quinoline Antimalarials

Hydroxychloroquine and chloroquine are active against SARS-CoV-2 in VERO cells but do not exhibit antiviral effect in animals. Moreover, randomized clinical trials of hydroxychloroquine and chloroquine do not support substantial clinical benefit in humans [Rosenke et al Hydroxychloroquine Proves Ineffective in Hamsters and Macaques Infected with SARS-CoV-2. bioRxiv. June 2020; Skipper et al Hydroxychloroquine in Nonhospitalized Adults With Early COVID-19: A Randomized Trial. Ann Intern Med. July 2020; Wang et al Remdesivir and chloroquine effectively inhibit the recently emerged novel coronavirus (2019-nCoV) in vitro. Cell Res. 2020; 30(3):269-271; Hoffman et al Chloroquine does not inhibit infection of human lung cells with SARS-CoV-2. Nature (2020) (Hoffman et al., 2020)] demonstrated that chloroquine and hydroxychloroquine were 10-fold less potent in the epithelial cell line CALU-3 than in VERO cells and in VERO cells expressing the protease TRMPSS2. Since SARS-CoV-2 is considered to enter human respiratory cells via a TRMPSS2-mediated mechanism rather than the low endosomal pH-dependent dependent mechanism important for entry into VERO cells, the clinical failure of hydroxychloroquine has been attributed to its inability to inhibit only the pH-dependent entry mechanism which is not present in human respiratory epithelial cells [because it acts by increasing host cell endosomal pH, see Hoffman et al 2020]. It has become dogma in the field that all quinoline antimalarials act in the same way and, thus, that all quinoline antimalarials will not work in epithelial cells or provide clinical benefit.

In vitro susceptibility data were generated for tafenoquine in the human endothelial cell line CALU3. As described herein, it was surprisingly found, especially given the literature, that tafenoquine exhibits useful inhibition of antiviral replication at pharmacologically achievable concentrations.

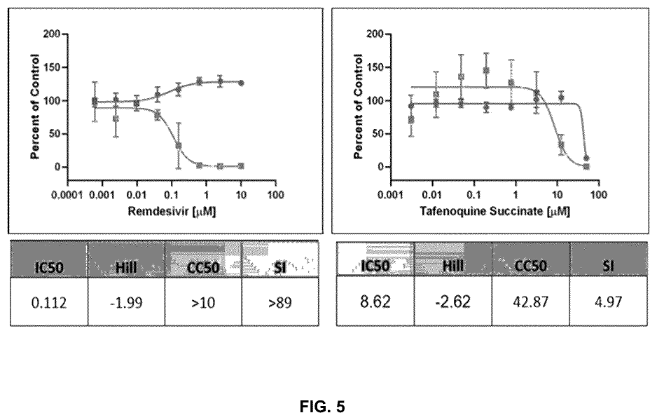

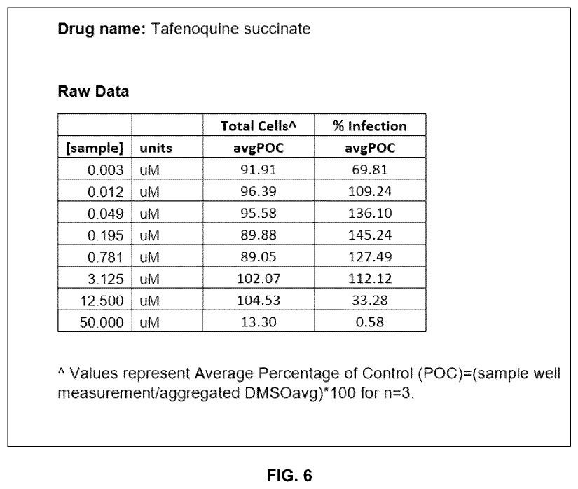

The EC50 of [same as the IC50 referred to in FIG. 5] tafenoquine against SARS-CoV-2 in CALU-3 cells was determined as described briefly in FIG. 5 and in detail as described by Dittmar et al., Drug repurposing screens reveal FDA approved drugs active against SARS-Cov-2. bioRxiv. June 2020 (Dittmar et al., 2020), and found to be 8.6 microM. The raw data are in FIG. 6. The cell culture medium used included 10% fetal bovine serum. Results showed that tafenoquine was more potent than any other quinoline antimalarial screened by Dittmar et al., 2020 in CALU3 cells.

The teachings of all patents, published applications, and references cited herein are incorporated by reference in their entirety for all purposes.

While this invention has been particularly shown and described with references to the example embodiments thereof, it will be understood by those skilled in the art that various changes in form and details may be made therein without departing from the scope of the invention encompassed by the appended claims.

1.-8. (canceled)

9. A method for treating or preventing a disease or infection with MCP-1 upregulation, or a symptom thereof, in a human subject, said method comprising:
(a) administering to said subject an effective amount of tafenoquine, a pharmaceutically-acceptable salt thereof, or a pharmaceutical composition comprising a compound of tafenoquine, and
and wherein said lung infection is caused by at least one virus.

10. The method according to claim 9, further comprising determining said subject has said disease or infection with MCP-1 upregulation or is at risk of contracting said disease or infection with MCP-1 upregulation.

11. (canceled)

12. The method according to claim 9, wherein the subject is glucose-6-phosphate dehydrogenase (G6PD)-normal.

13.-16. (canceled)

17. The method according to claim 9, wherein said administration is via sub-lingual and/or buccal and/or intravenous route(s).

18.-19. (canceled)

20. The method according to claim 9, wherein about 100 mg to about 600 mg is administered in one or more initial dose(s).

21. The method according to claim 9, wherein about 100 mg to about 600 mg is administered in one or more initial dose(s) and in one or more subsequent dose(s).

22. The method according to claim 9, wherein three initial doses are administered once per day for three days.

23. The method according to claim 9, wherein three or four initial doses are administered.

24. The method according to claim 21, wherein the subsequence dose(s) is administered once per week.

25. The method according to claim 21, wherein the subsequence dose(s) is administered once per day.

26. The method according to claim 20, wherein the initial dose(s) is about 200 mg.

27. The method according to claim 20, wherein the initial dose(s) is about 150 mg.

28. The method according to claim 20, wherein the initial dose(s) is about 100 mg.

29. The method according to claim 21, wherein the subsequent dose(s) is about 200 mg.

30. The method according to claim 21, wherein the subsequent dose(s) is about 150 mg.

31. The method according to claim 21, wherein the subsequent dose(s) is about 100 mg.

32. The method according to claim 21, wherein the first subsequent dose is administered seven days after the last initial dose.

33. The method according to claim 21, wherein the initial doses is about 200 mg and is administered once a day for three days, and wherein the subsequent dose(s) is about 200 mg and is administered once a week.

34.-35. (canceled)

36. The method according claim 9, wherein the disease or infection is gastric ulcer, bowel disease, diabetes, breast carcinoma, triple negative breast cancer, eosophageal squamous cell carcinoma, bacteria or fungi-associated respiratory tract infections, Alzheimer's disease, Parkinson's disease, multiple sclerosis, osteoarthririts, rheumatoid arthritis, osteoporosis, diseases related to endothelial cell dysfunction, lupus nephritis, COVID-19, SARS-CoV-2, a disease cause by SARS-CoV-2 with COVID-19-like symptoms persisting for more than 28 days, influenza, respiratory syncytial virus, Lyme disease, chronic fatigue syndrome and/or fibromyalgia; and/or wherein the condition being treated are adverse events associated with m-RNA vaccination for COVID-19 or other diseases.

37. A kit comprising: (a) a means for testing for G6PD deficiency; (b) a compound of Formula (I), a pharmaceutically-acceptable salt thereof, or a pharmaceutical composition comprising a compound of Formula (I); and 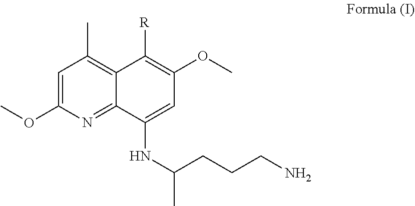
(c) instructions for use,
wherein R is any halogen-containing substituent of molecular weight ≤205.

38.-56. (canceled)
